(19) United States
(12) Patent Application Publication
(10) Pub. No.: US 2024/0312328 A1
(43) Pub. Date: Sep. 19, 2024

(54) SYSTEM AND METHOD FOR FALL DETECTION AND COMMUNITY-BASED MOBILITY IMPROVEMENT

(71) Applicant: OCHSNER CLINIC FOUNDATION d/b/a OCHSNER HEALTH, New Orleans, LA (US)

(72) Inventors: Richard MILANI, New Orleans, LA (US); Jonathan WILT, New Orleans, LA (US); Matthew HERZOG, New Orleans, LA (US)

(21) Appl. No.: 18/607,250

(22) Filed: Mar. 15, 2024

Related U.S. Application Data
(60) Provisional application No. 63/452,941, filed on Mar. 17, 2023.

(51) Int. Cl. G08B 21/04; G08B 25/01; G16H 10/20; G16H 20/00; G16H 50/20

(52) U.S. Cl. CPC G08B 21/043; G08B 21/0446; G08B 25/016; G16H 10/20; G16H 20/00; G16H 50/20

(57) ABSTRACT

A system and method that use a personal emergency response system (PERS) for fall detection and improving mobility is disclosed. The system and method for fall detection includes a PERS that has movement sensors incorporated therein configured to generate movement profiles of a user. A control system receives a movement profile from a movement sensor, compares a movement profile to a fall detection threshold indicative of a fall-down motion, and provides to the PERS, a notification prompting the user to activate an emergency contact function upon detecting that the movement profile met the fall detection threshold. The system and method for improving mobility includes a database storing user gait information and a control system configured to generate a mobility score of the user based on the gait information. Based on the mobility score of the user, the control system transmits a mobility improvement program to a device of the user.

### CROSS-REFERENCE TO RELATED APPLICATIONS

This application claims priority to U.S. Provisional Application No. 63/452,941, filed Mar. 17, 2023, the entire disclosure of which is incorporated herein by reference.

### FIELD OF THE INVENTION

The present invention relates generally to a system and method for fall prevention, and more specifically, to a system and method for fall detection and a platform for helping a user improve mobility by matching the user with a user support group comprising one or more other users. Add virtual coach enabled, and personalized interventions based on (a) sensor-based AI/ML and (b) advanced analytics derived from a database of similar uses that comprise “clinical doubles” for the user.

### BACKGROUND OF THE INVENTION

More than one in four older people fall each year, representing the second leading cause of unintentional injury deaths worldwide, and the leading cause of trauma-related hospitalizations among older adults in the United States. According to the Centers for Disease Control (CDC), an estimated 3 million emergency department (ED) visits, more than 950,000 hospitalizations or transfers to another facility, and approximately 32,000 deaths result from falls in older adults annually, representing the third most expensive health condition for this age group with hospitalization costs averaging $34,294.

While nearly 90 percent of older adults want to live in their residence for as long as possible, falls can rapidly lead to a loss of their desired independence. The risk of long-term admission to a nursing home rise from a three-fold to a five-fold increase following either a single or multiple non-injurious falls respectively, and to a 10-fold increase after one or more falls that result in serious injury. Because of the devastating impact that falls have on health, independence, and quality of life, older adults rate fear of falling highest relative to other common fears including criminal violence and financial crisis.

Most fall management strategies rely solely on the use of personal emergency response systems (PERS) that can provide a lifeline to an isolated adult following a fall. Although conventional PERS can reduce hospital length of stay, subscribers don't consistently wear them, often due to the negative stigma associated with it, and frequently do not summon emergency medical services (EMS) following a significant fall. This is in part due to the “all-or-none” response of engaging local EMS and dispatching an ambulance, where many individuals are unsure of whether emergency transportation to a hospital is appropriate for their circumstance. Rather, having immediate medical advice to provide guidance, as an additional layer of response short of EMS activation, was identified by Apple as an important supplement to existing PERS technology utilized in its Apple Watch (R. Milani, personal communication 2021).

Since an estimated 60% of falls in older adults are a result of multiple factors, the US Preventive Services Task Force (USPSTF), the American and British Geriatrics Societies, and other systematic reviews have recommended that any fall prevention program be capable of addressing each of the many components that contribute to the risk of falls.

Because of the shortcomings identified in existing fall management designs, there is a need to design a fall management system that intelligently allows a control system to determine an appropriate response after a falling motion has been detected by the user's PERS. Additionally, there is a need to utilize the valuable data provided by the PERS to more accurately monitor and help improve a user's mobility health.

### SUMMARY OF THE INVENTION

The term embodiment and like terms, e.g., implementation, configuration, aspect, example, and option, are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, not this summary. This summary is a high-level overview of various aspects of the disclosure and introduces some of the concepts that are further described in the Detailed Description section below. This summary is not intended to identify key or essential features of the claimed subject matter. This summary is also not intended to be used in isolation to determine the scope of the claimed subject matter. The subject matter should be understood by reference to appropriate portions of the entire specification of this disclosure, any or all drawings, and each claim.

According to some implementations of the present disclosure, a system for fall detection includes a personal emergency response system (PERS), a memory containing machine readable medium comprising machine executable code, and a control system. The PERS has at least one movement sensor incorporated into the PERS configured to generate one or more movement profiles of a first user. The control system coupled to the memory and is configured to execute the machine executable code to cause the control system to receive a first movement profile of the one or more movement profiles from the movement sensor. The control system is further configured to compare the first movement profile to a fall detection threshold indicative of a fall-down motion. The control system is further configured to, upon detecting that the first movement profile met the fall detection threshold, provide, to the PERS, a first notification prompting the first user to activate an emergency medical service (EMS) function.

According to some implementations of the present disclosure, a method for fall detection includes receiving a first movement profile of one or more movement profiles of a first user from at least one movement sensor. The method further includes comparing the first movement profile to a fall detection threshold indicative of a fall-down motion. The method further includes, upon detecting that the first movement profile has met the fall detection threshold, providing, to a personal emergency response system (PERS), a first notification prompting the first user to activate an emergency medical service (EMS) function.

According to some implementations of the present disclosure, a system includes a database and a control system. The database is configured to store gait information associated with a plurality of users including a first user. The control system is communicatively coupled to the database, wherein the control system is configured to generate a mobility score of the first user based on information including sensor data collected from a device associated with a first user among a plurality of users, a personal trait associated with the first user, or an environmental factor associated with the first user; generate a mobility improvement program for the first user based on the mobility score of the first user; and cause transmission of the mobility improvement program of the first user to a user device associated with the first user.

According to some implementations of the present disclosure, a method includes generating, by a control system, a mobility score of the first user based on information including sensor data collected from a device associated with a first user among a plurality of users, a personal trait associated with the first user, or an environmental factor associated with the first user. The method also includes generating, by the control system, a mobility improvement program for the first user based on the mobility score of the first user. The mobility improvement program can include one or more behavioral and environmental recommendations to the first user. The method further includes causing, by the control system, transmission of the mobility improvement program of the first user to a user device associated with the first user.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims. Additional aspects of the disclosure will be apparent to those of ordinary skill in the art in view of the detailed description of various embodiments, which is made with reference to the drawings, a brief description of which is provided below.

### BRIEF DESCRIPTION OF THE DRAWINGS

The present disclosure, and its advantages, will be better understood from the following description of representative embodiments together with reference to the accompanying drawings. These drawings depict only representative embodiments, and are therefore not to be considered as limitations on the scope of the various embodiments or claims.

FIG. 1 is an exemplary architecture of a system for fall detection, according to some implementations of the present disclosure.

FIG. 2A illustrates an exemplary user interface of a personal emergency response system (e.g., smartphone), according to some implementations of the present disclosure.

FIG. 2B illustrates another exemplary user interface of a personal emergency response system, according to some implementations of the present disclosure.

FIG. 3 is a flow diagram depicting example operations for fall detection, according to some implementations of the present disclosure.

FIG. 4A is a flow diagram depicting example operations for fall detection side view of a pinned end of the latch mechanism of FIG. 4A with the latch mechanism in the locked position, according to some implementations of the present disclosure.

FIG. 4B is a continuation of the flow diagram of FIG. 4A.

FIG. 4C is a continuation of the flow diagram of FIG. 4A.

### DETAILED DESCRIPTION

Various embodiments are described with reference to the attached figures, where like reference numerals are used throughout the figures to designate similar or equivalent elements. The figures are not necessarily drawn to scale and are provided merely to illustrate aspects and features of the present disclosure. Numerous specific details, relationships, and methods are set forth to provide a full understanding of certain aspects and features of the present disclosure, although one having ordinary skill in the relevant art will recognize that these aspects and features can be practiced without one or more of the specific details, with other relationships, or with other methods. In some instances, well-known structures or operations are not shown in detail for illustrative purposes. The various embodiments disclosed herein are not necessarily limited by the illustrated ordering of acts or events, as some acts may occur in different orders and/or concurrently with other acts or events. Furthermore, not all illustrated acts or events are necessarily required to implement certain aspects and features of the present disclosure.

For purposes of the present detailed description, unless specifically disclaimed, and where appropriate, the singular includes the plural and vice versa. The word “including” means “including without limitation.” Moreover, words of approximation, such as “about,” “almost,” “substantially,” “approximately,” and the like, can be used herein to mean “at,” “near,” “nearly at,” “within 3-5% of,” “within acceptable manufacturing tolerances of,” or any logical combination thereof. Similarly, terms “vertical” or “horizontal” are intended to additionally include “within 35% of” a vertical or horizontal orientation, respectively. Additionally, words of direction, such as “top,” “bottom,” “left,” “right,” “above,” and “below” are intended to relate to the equivalent direction as depicted in a reference illustration; as understood contextually from the object(s) or element(s) being referenced, such as from a commonly used position for the object(s) or element(s); or as otherwise described herein.

For the present disclosure, the terms “computer system” or “computer device” or “computing system” refer to any electronically-powered or battery-powered equipment that has hardware, software, and/or firmware components, where the software and/or firmware components can be configured for operating features on the device.

In some implementations, a system and method for fall detection is disclosed.

The present disclosure also discloses a system and method for fall management and prevention, which helps patients by generating a personalized mobility improvement program based on information collected from the user, such as gait information collected from a wearable device associated with the user and input from the user.

According to some implementations of the present disclosure, a fall management and prevention system includes an application (e.g., Connected Stability™) which can be installed on wearable devices such as smartphones or smartwatches. The system is for preventing injurious falls among people with compromised mobility, whether due to injury or infirmity. Notably, this system is particularly helpful to elderly users who live independently, possibly alone. Such elderly users often have a “Support Person,” typically an adult child, who serves as an informal, occasional caregiver, but they have historically been able to take care of their activities of daily living without assistance. As their mobility declines, for any number of reasons, the Support Person becomes concerned.

The system relies on wearable sensors for its connected elements. For example, the system can utilize a smartphone to provide gait analytics based on multiple factors. Additionally, or alternatively, the system can utilize a smartwatch or another piece of wearable device to provide fall detection and instant alerts.

According to some implementations, the application collects sensor data from one or more devices associated with a user (e.g., a wearable device or smartphone). The collected sensor data include data such as gait information of the user. Further, according to some embodiments, the application collects additional setup information from the user by asking the user a series of setup questions to take into account one or more behavioral and environmental factors that can have impact on the user's mobility health. Setup questions can include questions about personal traits of the user, such as age, gender, location, interests, hobbies, likes, dislikes, preferences, and health issues (e.g., co-morbidities). According to some embodiments, based on the collected sensor data, the application generates a “mobility score”. According to some embodiments, the mobility score is an index or a series of indices that assess how likely it is that the user can move freely and easily, without falling. The mobility score can be utilized to follow a user's mobility over time as well as track and quantify the impact of fall-prevention related interventions based on changes in the mobility score. For example, the mobility score can be configured to measure mobility on a plurality of factors including the user's steps count over a predefined period, balance and strength training measures, mobility hazards, medication impact on gait, and the quality of gait. The quality of gait and step count have been well-studied and normative data specific to age and sex of the user can all be used to generate the mobility score. According to additional embodiments, generation of the mobility score is further based on personal behavioral and environmental factors of the user that affect mobility. For example, information about such factors can be collected from the user through the previously described series of setup questions. As data accumulates from a population of users, the algorithm for calculating the mobility score can be revised so that the mobility score is both (1) a more accurate and personalized indicator of a user's current mobility health, and (2) can more predict which intervention plans will have the highest probability of enhancing the mobility health of the user. The mobility score is then used by the application to generate a personalized mobility improvement program to help the user improve mobility and reduce the likelihood of fall-induced injuries. According to some embodiments, the personalized mobility improvement program may include one or more of the following elements: (1) home hazard review; (2) training or physical therapy plan (e.g., balance and strength training schedule); (3) recommendations for foot health (e.g., pain and/or neuropathy), footwear and orthotics; (4) medication review and deprescription recommendation; (5) recommendation for nutritional intake (e.g., taking one or more supplements or nutraceuticals), and hydration; (6) urinary incontinence screening; (7) connectivity to a caregiver for support via a user device (e.g., computer, smartphone, or wearable device); (8) multi-variable gait analysis based on data collected from one or more wearable sensors; (9) predictive analytics model based on gait information to identify trends and create appropriate interventions; (10) automated, virtual “mobility coach” to provide appropriate guidance and improvement tasks; (11) “medical twin” model based on a composite of data from other similarly-situated users to determine which interventions will be most effective; (12) social connectivity to other users via a user device (e.g., a connected caregiver or one or more other users); (13) gamification of improvement tasks to encourage the user to continue participation in the improvement program; (14) recommendation for the user to wear of a PERS device; (15) access to a contact center that provides emergency medical services to the user; (16) data integration with one or more electronic medical record systems; and (17) adaptational changes to the user's home environment.

As an example of the implementation, a system includes a database and a control system that is communicatively coupled to the database. The database can be configured to store gait information associated with a plurality of users including a first user (e.g., the main elderly user). The control system is configured to: generate a mobility score of the first user based on the gait information, generate a mobility improvement program for the first user based on the mobility score of the first user, and cause transmission of the mobility improvement program of the first user to a user device associated with the first user (e.g., a smartphone, a smartwatch, a tablet, or a personal computer). According to some implementations, the gait information can be based on sensor data generated by one or more movement sensors of a wearable device associated a user. The types of wearable devices can include at least one of a PERS, a smartwatch, an activity tracker, a fitness tracker, a safety monitor, and a personal alarm device.

According to an implementation, the control system can add one or more modules to the user's mobility improvement program to help the user with balance and strength training. For example, based on the user's participation in the balance and strength training, the control system can calculate a change in the mobility score of the user and generate a notification to indicate the impact of the training on the mobility score of the user. This feedback can be used to encourage a given behavior, or to recommend an alternative intervention that will be more successful. For example, the notification can be displayed on a user device (e.g., a smartphone) on which the application is installed.

According to some implementations, the mobility improvement program is gamified to encourage compliance and long-term usage by the user. For example, the system can enter the first user into competition with other users or complete challenges. The first user can receive rewards such as badges (e.g., digital tokens received in the application) or recognitions (e.g., notifications displayed on the user device to indicate the user's accomplishments) for completing tasks, challenges, competitions, or making certain achievements. As an example of this feature being implemented, a control system can be configured to compare the mobility score of the first user to a mobility score of the one or more second users (e.g., other elderly users) to generate a comparison outcome and cause transmission of a notification indicative of the comparison outcome to the user device of the first user. Further, the first user may be able to share the outcome with other users through the control system or to a third-party platform such as a social media platform (e.g., Facebook, Twitter, Instagram, etc.).

According to some implementations, the control system can be configured to match the first user to a group of users based on affinity-such as having similar mobility score, length of time in the mobility improvement program, mobility/exercise goals, or other criteria. For example, some other types of criteria can include personal information about the user such as age, gender, location, interests, hobbies, likes, dislikes, and preferences. To implement the above feature, the control system can be configured to define a user group comprising one or more second users of the plurality of users, wherein the one of more second users share a common trait. Further, the control system can be configured to match the first user to the user group based on the first user having the common trait in the gait information or personal information of the first user. Once a matching user group has been identified, the control system can be further configured to cause transmission of a notification to a user device associated the with the first user, the notification including a recommendation for the first user to join the user group. Alternatively, or additionally, the control system can be configured to automatically enroll the first user into the first user group. According to some implementations, the control system creates a platform such a landing page in the application (e.g., Connected Stability™) where users of the user group can provide motivation to each other and share information, such as solicit/give advice and exchange experience.

According to some embodiments, the control system can also generate a composite mobility score constructed by artificial intelligence using the mobility score and activities of multiple real users. For example, the control system identifies, based on one or more factors, correlations between the first user and a subset of users from a pool of real users. According to some implementations, the subset of users can be other users who are similar to the first user, such as having similar mobility (i.e., mobility-matched). According to some implementations, the control system can generate the composite mobility score based on the mobility scores of the subset of users, cause transmission of the composite mobility score to the user device of the first user, and compare the mobility score of the first user to the composite mobility score. According to some embodiments, the control system uses artificial intelligence to generate a “medical twin” based on the composite mobility score. The medical twin is a virtual user with characteristics similar to the first user. The medical twin can be used by the control system or the first user to probe the effectiveness of one or more interventions on users with mobility similar to the first user and predict how likely an intervention plan is to be effective at improving mobility health of the first user. For example, the prediction can be based on the observed effect that an intervention plan has on one or more users forming the subset of users used to construct the medical twin. According to some embodiments, the control system uses the observed effect on the subset of users to extrapolate an effect that the intervention plan could have on the first user. Based on the outcome of the extrapolation, the control system can suggest one or more interventions to the first user.

According to some implementations, the control system can dynamically generate prediction models using an artificial intelligence system, such as using fall prediction algorithm based on data from sources such as the Clarity electronic medical record (EMR) database by Epic Systems Corporation. Detailed information on the predictive model and analysis methodology is available in the document titled “Reducing Falls in Older Adults Using Digital Tools,” which is incorporated into this application by reference. Using the fall prediction algorithm, the control system can be configured to identify subtle changes in the gait of the first user and provide appropriate preventive steps such as by providing notifications and dynamically updating the mobility improvement program. According to some embodiments, the control system can be further configured to match mobility patterns and co-morbidities of the first user using artificial intelligence to determine what interventive steps will most likely produce optimal outcomes. Furthermore, to increase compliance of the first user with the mobility improvement program, the control system can be configured to cause transmission of personalized notifications to the user device associated with the first user with messages such as feedback from other users like the first user.

According to some embodiments, the control system is further configured to send surveys to the first user via the user device to inquire about health information and cause transmission of recommendations (e.g., healthcare recommendations) to the user device associated with the first user based on information provided about the first user. For example, the control system may send to the user device a survey about foot health. Once a diagnostic survey is received about the first user's health condition, the control system can generate and cause recommendations to be transmitted to the user device such as recommendations to address foot pain, neuropathy, types of preferred footwear, and orthotics. Detailed information on the flow diagram of survey questions is disclosed in the document titled “Workflow-Fall Prevention,” which is incorporated into this application by reference.

According to some implementations, the system further includes a second application (e.g., LivWatch™) that pairs a user device associated with the first user with a user device associated with a support person, such as an adult child, who serves as an informal, occasional caregiver to the first user. For example, the control system can be configured to (1) send to the second application a home safety checklist that helps the support person inspect the user's home for safety hazards and upload images of the user's home to a server for home safety inspection; (2) receive from the second application a list of medication taken by the user, create a customized analysis of the medications, and generate deprescribing advice, which the first user or the support person could then take to a primary caregiver for review; (3) plan and schedule personalized rewards or special activity for the first user, which could be sent to the user device associated with the first user at a predetermined time or upon the first user reaching a milestone goal; and (4) send a weekly update to the second application to notify the support person of the status of the first user, such as the first user's level of engagement with the mobility improvement program and changes in the first user's mobility score.

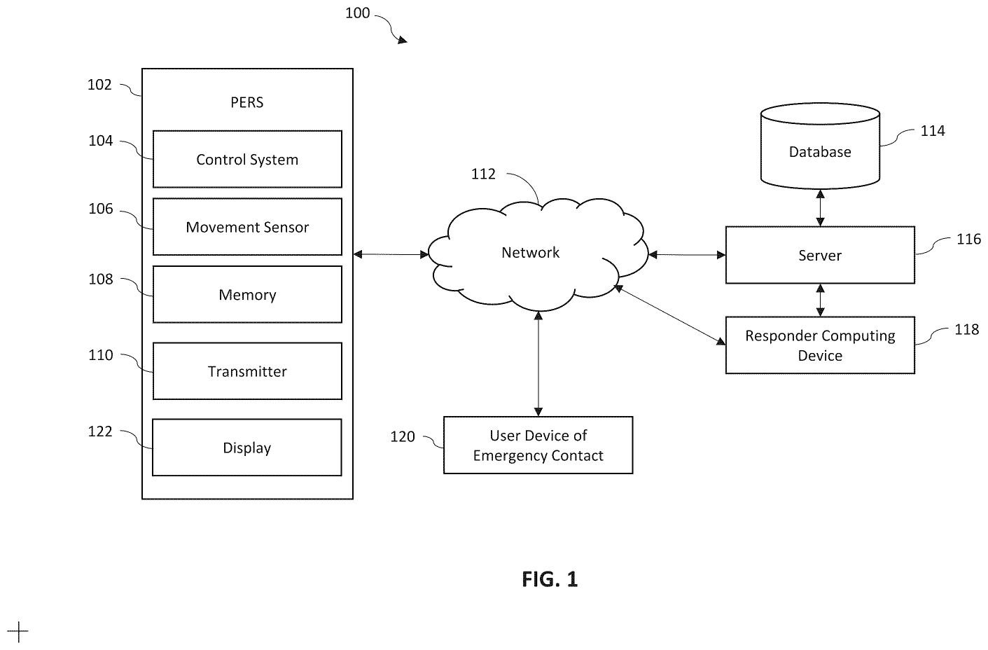

Turning now to FIG. 1, an exemplary block diagram of a system for fall detection is shown. According to some embodiments, the system 100 includes a personal emergency response system (PERS) 102, a memory 108 containing machine readable medium comprising machine executable code, a control system 104, one or more movement sensors 106 configured to generate one or more movement profiles of a first user, and one or more transmitters 110. The control system 104 is coupled to the memory 108 and is configured to execute the machine executable code to cause the control system 104 to receive a first movement profile of the one or more movement profiles from the one or more movement sensors 106. According to some embodiments, the control system is further configured to compare the first movement profile to a fall detection threshold indicative of a fall-down motion. Upon detecting that the first movement profile meets the fall detection threshold, the control system 104 provides, to the PERS 102, a first notification prompting the first user to activate an emergency medical service (EMS) function. According to some embodiments, the control system 104, the one or more movement sensors 106, the memory 108, and the transmitter 110 are incorporated into the PERS 102. For example, the PERS 102 may be a smartwatch with the foregoing components included in the smartwatch.

According to some embodiments, the system 100 may further include a database 114 for storing data associated one or more users including the first user, a server 116, and a responder computing device 118. The server 116 and the responder computer device 118 can be communicatively coupled to the PERS 102 via a network 112. For example, when the first movement profile meets the fall detection threshold, the control system 104 provides a first notification to the PERS prompting the first user whether to activate an emergency medical service (EMS) function. According to some embodiments, the first notification is shown on a display 122. Depending on the first user's selection or lack of selection, the control system is configured to send an alert status to the transmitter 110 and the network 112. Depending on the type of alert status that is registered in the server 116, the server initiates one or more corresponding types of response protocol on the responder computing device 118. According to some embodiments, the types of response protocol may include a series of automatic response or displaying on the responder computing device 118 a sequence of directions to be followed by the responder (e.g. call the first user to inquire whether an ambulance is needed. Details on the subject matter of the types of alert statuses and the protocols are further described below.

According to some embodiments, the system may include a second user device associated with a second user (e.g., an emergency contact listed for the first user in the database 114). According to some protocols triggered by the PERS 102, the responder computing device 118 can send notification or call the second user device 120 to notify the second user of fall detection event.

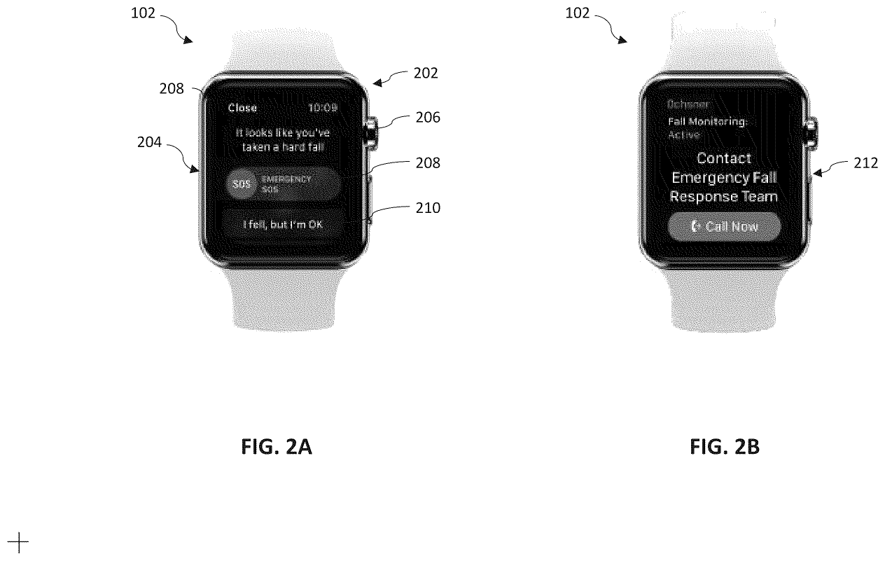

According to some embodiments, the PERS may include at least one of a wearable device, a smartwatch, an activity tracker, a fitness tracker, a safety monitor, and a personal alarm device. FIG. 2A and FIG. 2B illustrate an exemplary PERS 102 that is a smartwatch. FIG. 2A illustrates a user interface, according to an embodiment of the present disclosure. As described above, the control system (not shown in FIGS. 2A and 2B) receives a first movement profile from the movement sensor incorporated into the smartwatch 102. The control system compares the first movement profile to a fall detection threshold indicative of a fall-down motion. Upon detecting that the first movement profile has met the fall detection threshold, the control system provides, to the smartwatch 102, a first notification 204. According to some embodiments, the first notification 204 is shown on a digital display 202 of the smartwatch 102. The first notification 204 prompts the first user whether to activate an emergency medical service (EMS) function. In some embodiments, the first user can activate the EMS function by engaging a first virtual button 208 shown on the digital display 202. Engaging the first virtual button 208 (e.g., by swiping the first virtual button 208) causes the smartwatch to initiate communication with an EMS responder immediately (e.g., make phone call) and sends a first alert status to a server (e.g., a customer relation management server).

According to some embodiments, the smartwatch 102 also has a second virtual button 210 that allows the first user to indicate that he/she had fallen but is not injured. Engaging the second virtual button 210 (e.g., by tapping the second virtual button 210) would not cause the smartwatch to immediately call the EMS responder but will send an alert status to the server to indicate that the first user had a fall detection event. According to some embodiments, the alert status will cause the computing device of the responder (not shown in FIGS. 2A and 2B) to initiate a response protocol, which may instruct the EMS responder to contact and check on the status of the first user.

According to some embodiments, the digital display 202 may further show a “close” button 208, engaging which causes the smartwatch 102 to decline to contact the EMS. According to some embodiments, a digital crown 206 of the smartwatch 102 can also be configured such that tapping on the digital crown 206 also causes the smartwatch 102 to decline to contact the EMS. It should be noted that it would be apparent to a person of ordinary skill in the art that the above-described can be buttons can be substituted by other types digital or physical components that perform the same function.

FIG. 2B shows a second user interface 212 of the smartwatch 102. In the event that the first user engages the first virtual button to contact the EMS responder, the smartwatch 102 would call the EMS responder. The user display 202 of the smartwatch 202 changes to the second user interface 212 to show that it is contacting the EMS responder.

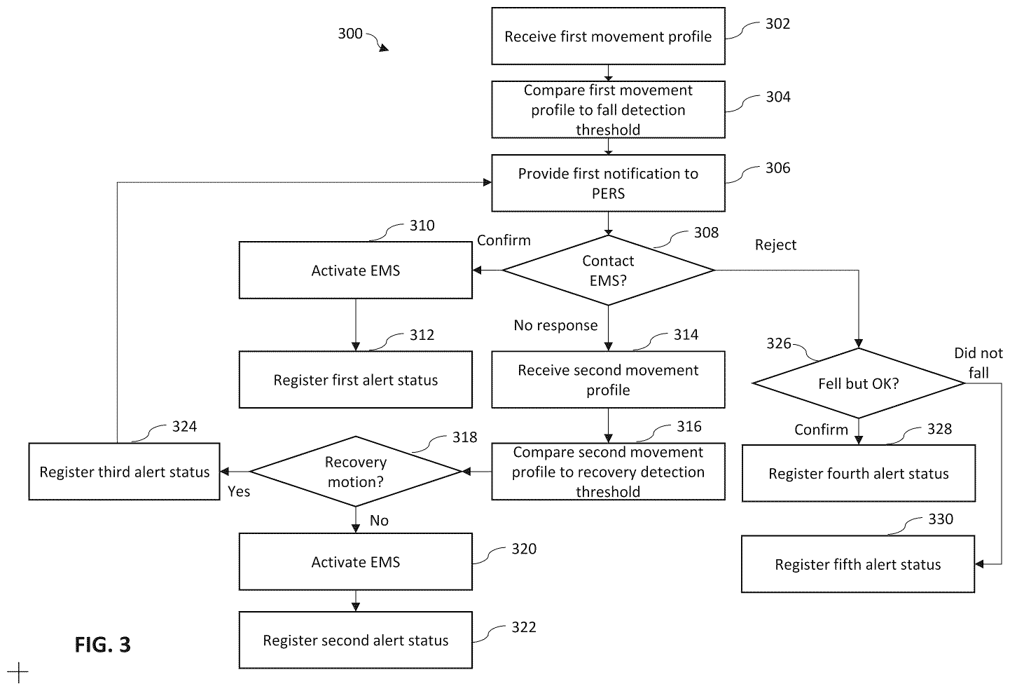

FIG. 3 is a flow diagram 300 depicting example operations for fall detection, according to some implementations of the present disclosure. The flow begins at block 302.

At block 302, the first movement profile is received. For example, the PERS has one or more movement sensors incorporated into the PERS configured to generate one or more movement profiles of a first user; the control system receives a first movement profile from the one or more movement sensors.

At block 304, the first movement profile is compared to the fall detection threshold. For example, the control system compares the first movement profile to a fall detection threshold indicative of a fall-down motion. The fall detection threshold can include fall detection algorithm embedded in the PERS.

At block 306, the first notification is provided to the PERS. For example, upon detecting that the first movement profile has met the fall detection threshold, the control system provides, to the PERS, a first notification prompting the first user whether to activate an emergency medical service (EMS) function. For example, the fall detection threshold could include the one or more of the movement sensors detecting one or a combination of a sudden drop and stop in altitude, a sudden tilt motion, a vibration, or any other types of movement profile indicative of a fall.

At block 308, it is determined whether to contact the EMS. For example, the first user can input a selection on his/her PERS that determines whether to activate EMS. An exemplary first notification sent to the user interface of a smartwatch is shown. For example, the first notification includes the option for the first user to select “SOS”.

At block 310, the EMS is activated. For example, by swiping right on the “SOS” button, the first user is able to activate EMS. According to some implementations, activating EMS will initiate response protocol involving one or more of a medical team, a service center agent, and the support person paired with the first user.

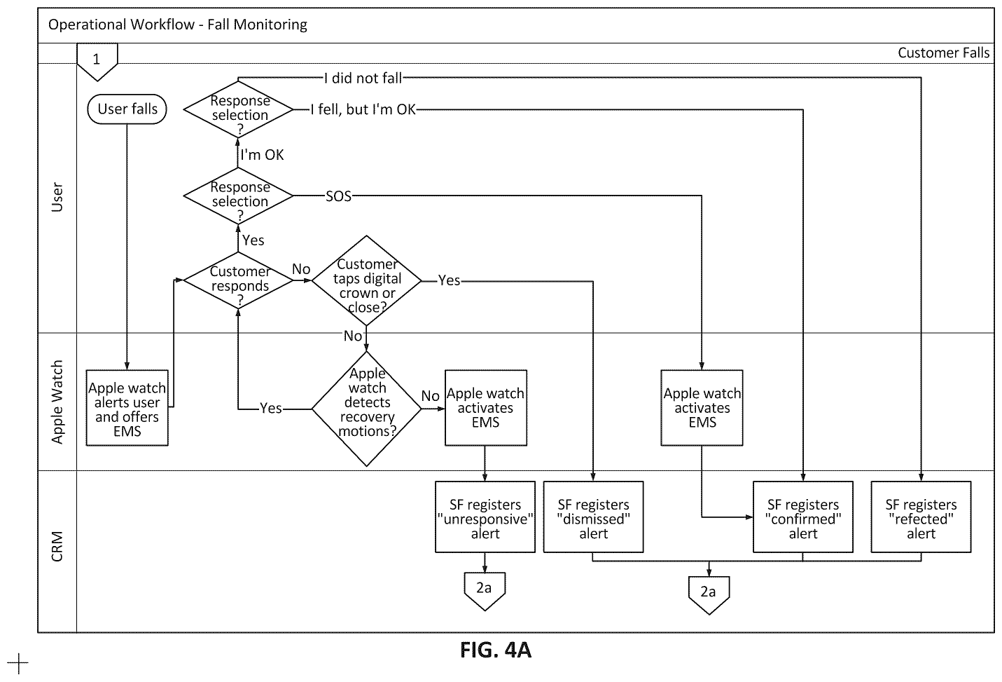

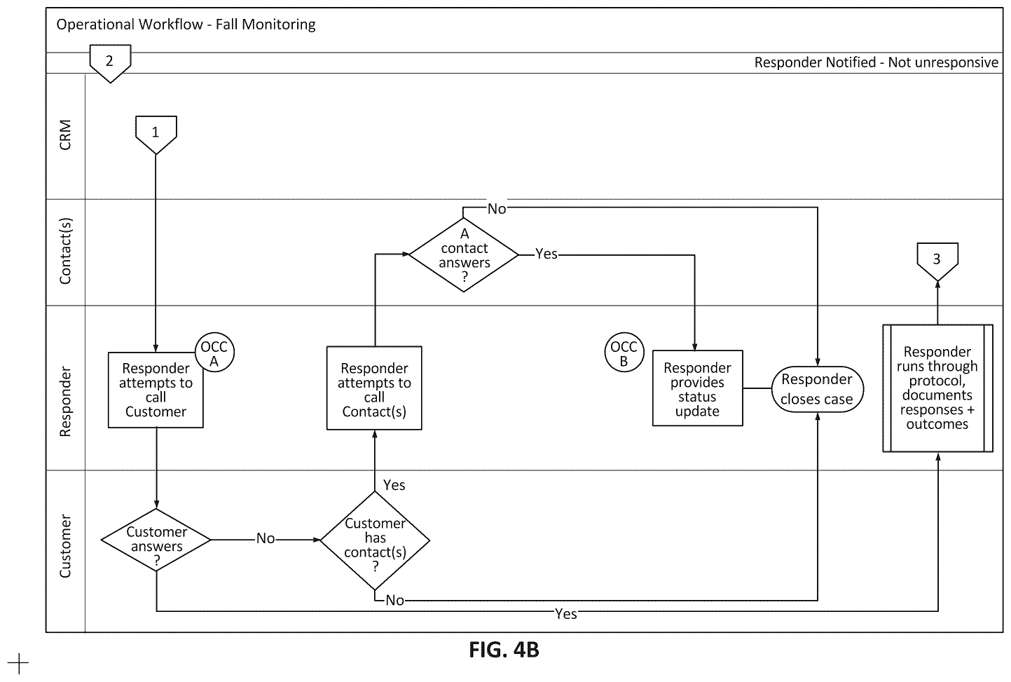

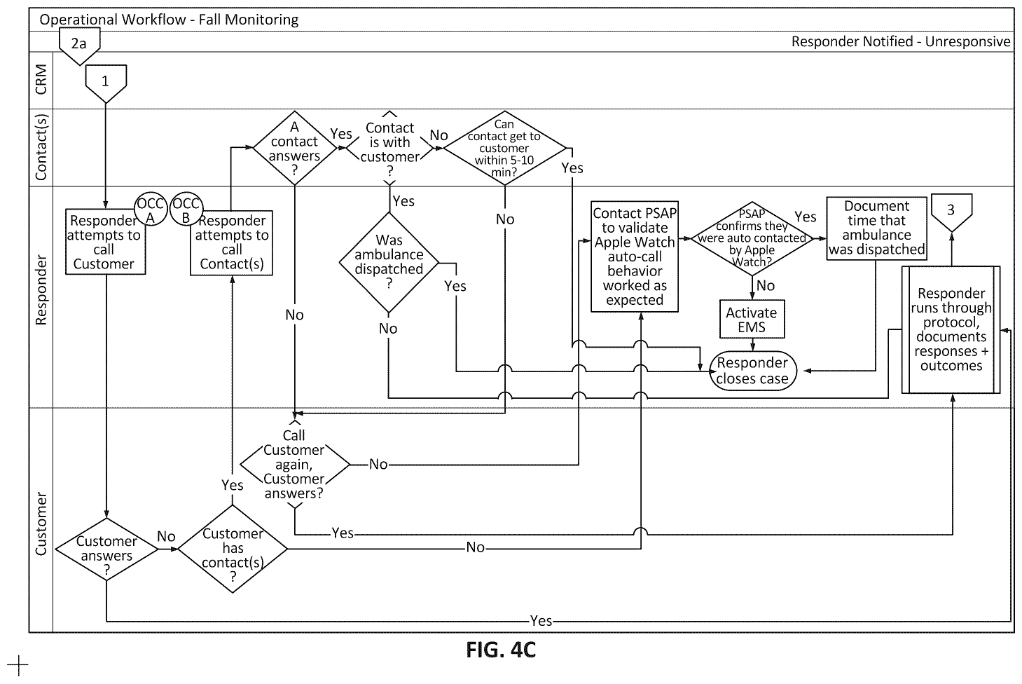

At block 312, the first alert status is registered. For example, according to some implementations, after activating EMS, the control system causes a customer relationship management (CRM) system to register a first alert status in the database. The first alert status instructs the CRM system to initiate a response protocol. An exemplary protocol is shown in FIGS. 4A and 4B. Detailed information on the response protocol is available in the document titled “Workflow-Fall Detection”, which is incorporated into this application by reference.

At block 314, a second movement profile is received. For example, the control system is further configured to receive a second movement profile of the one or more movement profiles from the PERS. If the control system does not receive a response to the first notification within a predefined time threshold, the control system compares the second movement profile to a recovery detection threshold indicative of a recovery motion to determine if the first user has recovered from the fall.

At block 316, the second movement profile is compared to a recovery detection threshold. For example, the control system compares, or causes the first user's smartphone to compare, the second movement profile with the recovery detection threshold.

At block 318, whether a recovery has been detected is determined. For example, the control system determines based on the comparison of the second movement profile and the recovery detection threshold whether the first user has recovered from the fall.

At block 320, the EMS is activated. For example, if the control system determines that the second movement profile does not meet the recovery detection threshold, which is an indication that the first user did not recover from the fall, the control system will cause the user device (e.g., smartwatch) to activate EMS.

At block 322, a second alert status is registered. For example, when the control system or the smartwatch does not detect a second movement profile that passes the recovery detection threshold, the control system can further register a second alert status. According to some implementations, the control system causes the CRM system to register a second alert status in the database. The second alert status instructs the CRM system to initiate a corresponding response protocol. An exemplary protocol is shown in FIGS. 4A and 4C. Detailed information on the response protocol is available in the document titled “Workflow—Fall Detection.

At block 324, a third alert status is registered. For example, if the control system or the smartwatch detects a second movement profile (i.e., a recovery motion) that passes the recovery motion threshold, the control system can send a third alert status to the control system causes the CRM system to register a third alert status in the database. The third alert status instructs the CRM system to initiate a corresponding response protocol. According to some implementations, the protocol triggered by the third alert status is identical to the protocol triggered by the first alert status.

At block 326, a status of whether the first user fell but is uninjured is determined. For example, returning to block 308, the first notification can include an button for an option such as “I fell, but I'm OK”.

At block 328, a first alert status is registered. For example, if the first user confirms “I fell, but I'm OK” by selecting the button, the control system causes the CRM system to register a fourth alert status in the database. The fourth alert status instructs the CRM system to initiate a corresponding response protocol. According to some implementations, the protocol triggered by the fourth alert status is identical to the protocol triggered by the first alert status.

At block 330, a fifth alert status is registered. For example, if the first user selects the icon “Close” or touch the crown of the smartwatch, the first user can indicate that he/she did not fall. When the control system receives indication from the first user that he/she did not fall (e.g., a false alarm), the control system causes the CRM system to register a fifth alert status in the database. The fifth alert status instructs the CRM system to initiate a corresponding response protocol.

Referring now to FIGS. 4A through 4C, a flow diagram depicting example operations for fall depiction with inclusion of the protocols initiated by the alert statuses is depicted. The flow diagrams in FIGS. 4A to 4C further indicate the actors who will perform each of the steps in a response protocol. Detailed information on the response protocol is available in the document titled “Workflow-Fall Detection”, which is incorporated into this application by reference.

The implementations described above for FIGS. 1 to 4C are primarily in the context of a fall detection system and method implemented with a PERS such as a smartphone or a smartwatch. However, the described fall detection system and method are applicable to other types personal computing devices or wearable devices. The described system and method for fall detection have been presented by way of example only, and not limitation, and can include different combinations of the described elements.

Although the disclosed embodiments have been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

While various embodiments of the present disclosure have been described above, it should be understood that they have been presented by way of example only, and not limitation. Numerous changes to the disclosed embodiments can be made in accordance with the disclosure herein, without departing from the spirit or scope of the disclosure. Thus, the breadth and scope of the present disclosure should not be limited by any of the above-described embodiments. Rather, the scope of the disclosure should be defined in accordance with the following claims and their equivalents.

What is claimed is:

1. A system for fall detection, the system comprising:
a personal emergency response system (PERS), the PERS having at least one movement sensor incorporated into the PERS configured to generate one or more movement profiles of a first user;
a memory containing machine readable medium comprising machine executable code having stored thereon instructions; and
a control system coupled to the memory comprising one or more processors, the control system configured to execute the machine executable code to cause the control system to:
receive a first movement profile of the one or more movement profiles from the movement sensor;
compare the first movement profile to a fall detection threshold indicative of a fall-down motion;
upon detecting that the first movement profile has met the fall detection threshold, provide, to the PERS, a first notification prompting the first user whether to activate an emergency medical service (EMS) function.

2. The system of claim 1, wherein the control system is further configured to:
upon receiving a confirmative response to the first notification, activate the EMS function.

3. The system of claim 2, wherein the control system is further configured to:
upon receiving a confirmative response to the first notification, cause a server database to register a first alert status, wherein the server database is in communicative contact with the control system.

4. The system of claim 3, wherein the first alert status being registered causes a computing device associated with a responder to execute a first response protocol.

5. The system of claim 1, wherein the control system is further configured to:
receive a second movement profile of the one or more movement profiles from the PERS; and
upon not receiving a response to the first notification within a predefined time threshold, compare the second movement profile to a recovery detection threshold indicative of a recovery motion.

6. The system of claim 5, wherein the control system is further configured to:
if the second movement profile does not meet the recovery detection threshold, activate the EMS function.

7. The system of claim 5, wherein the control system is further configured to:
if the second movement profile does not meet the recovery detection threshold, cause a server database to register a second alert status, wherein the server database is in communicative contact with the control system.

8. The system of claim 7, wherein the second alert status being registered causes a computing device associated with a responder to execute a second response protocol.

9. The system of claim 1, wherein the PERS includes at least one of a wearable device, a smartwatch, an activity tracker, a fitness tracker, a safety monitor, and a personal alarm device.

10. The system of claim 1, wherein the control system is further configured to:
upon receiving a confirmative response to the first notification, cause a server database to register a first alert status, wherein the server database is in communicative contact with the control system.

11. A method for fall detection, the method comprising:
receiving a first movement profile of one or more movement profiles of a first user from at least one movement sensor;
comparing the first movement profile to a fall detection threshold indicative of a fall-down motion; and
upon detecting that the first movement profile has met the fall detection threshold, providing, to a personal emergency response system (PERS), a first notification prompting the first user to whether activate an emergency medical service (EMS) function.

12. The method of claim 11 further comprising:
upon receiving a confirmative response to the first notification, activating the EMS function.

13. The method of claim 12 further comprising:
upon receiving a confirmative response to the first notification, cause a server database to register a first alert status, wherein the server database is in communicative contact with the control system.

14. The method of claim 13, wherein the first alert status being registered causes a computing device associated with a responder to execute a first response protocol.

15. The method of claim 11 further comprising:
receiving a second movement profile of the one or more movement profiles from the PERS; and
upon not receiving a response to the first notification within a predefined time threshold, comparing the second movement profile to a recovery detection threshold indicative of a recovery motion.

16. The method of claim 15 further comprising:
if the second movement profile does not meet the recovery detection threshold, activating the EMS function.

17. The method of claim 15 further comprising:
if the second movement profile does not meet the recovery detection threshold, causing a server database to register a second alert status, wherein the server database is in communicative contact with the control system.

18. The method of claim 17, wherein the second alert status being registered causes a computing device associated with a responder to execute a second response protocol.

19. The method of claim 11, wherein the PERS includes at least one of a wearable device, a smartwatch, an activity tracker, a fitness tracker, a safety monitor, and a personal alarm device.

20. The method of claim 1, wherein the control system is further configured to: upon receiving a confirmative response to the first notification, cause a server database to register a first alert status, wherein the server database is in communicative contact with the control system.

21. A system comprising:
a database configured to store gait information associated with a plurality of users including a first user; and
a control system communicatively coupled to the database, wherein the control system is configured to:
generate a mobility score of the first user based on the gait information;
generate a mobility improvement program for the first user based on the mobility score of the first user; and
cause transmission of the mobility improvement program of the first user to a user device associated with the first user.

22. The system of claim 21, wherein the gait information of each of the plurality of users is based on sensor data generated by one or more movement sensors of a wearable device associated with each of the plurality of users.

23. The system of claim 21, wherein the wearable device includes at least one of a personal emergency response system, a smartwatch, an activity tracker, a fitness tracker, a safety monitor, and a personal alarm device.

24. The system of claim 21, wherein generation of the mobility score is further based on user response to one or more setup questions directed to the first user.

25. The system of claim 21, wherein content of the mobility improvement program includes at least one of home hazard review, balance training program, strength training program, foot health recommendation, footwear recommendation, orthotics recommendation, medication review and deprescription recommendation, nutritional recommendation, urinary incontinence screening, connectivity to a caregiver, gait analysis, mobility improvement guidance, task, wearable device recommendation, user group recommendation, and a predictive analytics model to identify health trends of the first user.

26. The system of claim 21, wherein the control system is further configured to:
generate a predictive analytics model of one or more mobility health trends of the first user based on gait information of the first user.

27. The system of claim 21, wherein the control system is further configured to:
define a user group comprising one or more second users of the plurality of users,
wherein the one of more second users share a common trait; and
match the first user to the user group based on the first user having the common trait in the gait information or personal information of the first user.

28. The system of claim 27, wherein the control system is further configured to:
cause transmission of a notification to a user device associated the with the first user, the notification including a recommendation for the first user to join the user group.

29. The system of claim 27, wherein the control system is further configured to: automatically enroll the first user into the first user group.

30. The system of claim 27, wherein the personal information associated with the plurality of users includes one or more of age, gender, location, interests, hobbies, likes, dislikes, preferences, and health issues.

31. The system of claim 27, wherein the control system is further configured to:
compare the mobility score of the first user to a mobility score of the one or more second users to generate a comparison outcome; and
cause transmission of a notification indicative of the comparison outcome to the user device of the first user.

32. The system of claim 21, wherein the control system is further configured to:
generate a composite mobility score based on the mobility scores of a subset of users of the plurality of users;
cause transmission of the composite mobility score to the user device of the first user; and
compare the mobility score of the first user to the composite mobility score.

33. The system of claim 32, wherein the control system is further configured to:
generate a virtual user having the composite mobility score;
predict effectiveness of one or more intervention strategies based on simulated effect of the one or more intervention strategies on the virtual user; and
share with the first user at least one of the one or more intervention strategies that is most effective at increasing mobility of the virtual user.

34. The system of claim 21, wherein the control system is further configured to:
generate a second mobility score of the first user based on the patient health information of the first user from a second point in time.

35. A method comprising:
generating, by a control system, a mobility score of the first user based on gait information associated with a plurality of users including the first user;
generating, by the control system, a mobility improvement program for the first user based on the mobility score of the first user; and
causing, by the control system, transmission of the mobility improvement program of the first user to a user device associated with the first user.

36. The method of claim 35, wherein the gait information of each of the plurality of users is based on sensor data generated by one or more movement sensors of a wearable device associated with each of the plurality of users.

37. The method of claim 35, wherein the wearable device includes at least one of a personal emergency response system, a smartwatch, an activity tracker, a fitness tracker, a safety monitor, and a personal alarm device.

38. The method of claim 35, wherein generation of the mobility score is further based on user response to one or more setup questions directed to the first user.

39. The method of claim 35, wherein content of the mobility improvement program includes at least one of home hazard review, balance training program, strength training program, foot health recommendation, footwear recommendation, orthotics recommendation, medication review and deprescription recommendation, nutritional recommendation, urinary incontinence screening, connectivity to a caregiver, gait analysis, mobility improvement guidance, task, wearable device recommendation, user group recommendation, and a predictive analytics model to identify health trends of the first user.

40. The method of claim 35 further comprising:
generating, by the control system, a predictive analytics model of one or more mobility health trends of the first user based on gait information of the first user.

41. The method of claim 35 further comprising:
defining, by the control system, a user group comprising one or more second users of the plurality of users, wherein the one of more second users share a common trait; and
matching, by the control system, the first user to the user group based on the first user having the common trait in the gait information or personal information of the first user.

42. The method of claim 41 further comprising:
causing, by the control system, transmission of a notification to a user device associated the with the first user, the notification including a recommendation for the first user to join the user group.

43. The method of claim 41, wherein the control system is further configured to: automatically enroll the first user into the first user group.

44. The method of claim 41, wherein the personal information associated with the plurality of users includes one or more of age, gender, location, interests, hobbies, likes, dislikes, preferences, and health issues.

45. The method of claim 41 further comprising:
comparing, by the control system, the mobility score of the first user a mobility score of the one or more second users to generate a comparison outcome; and
causing, by the control system, transmission of a notification indicative of the comparison outcome to the user device of the first user.

46. The method of claim 35 further comprising:
generating, by the control system, a composite mobility score based on the mobility scores of a subset of users of the plurality of users;
causing, by the control system, transmission of the composite mobility score to the user device of the first user; and
comparing, by the control system, the mobility score of the first user to the composite mobility score.

47. The method of claim 46, wherein the control system is further configured to:
generating, by the control system, a virtual user having the composite mobility score;
predicting, by the control system, effectiveness of one or more intervention strategies based on simulated effect of the one or more intervention strategies on the virtual user; and
causing, by the control system, transmission of a recommendation to the user device associated with the first user of at least one of the one or more intervention strategies that is most effective at increasing mobility of the virtual user.

48. The method of claim 21 further comprising:
generating, by the control system, a second mobility score of the first user based on the patient health information of the first user from a second point in time.
